(19) United States
(12) Patent Application Publication
(10) Pub. No.: US 2016/0175173 A1
(43) Pub. Date: Jun. 23, 2016

(54) PORTABLE PERSONAL HYGIENE PRODUCTS HOLDER

(71) Applicant: FLO'S FRIENDS, LLC, Kathleen, GA (US)

(72) Inventors: Lanita M. Higgs-Jackson, Kathleen, GA (US); Chiquita B. Porter, Byron, GA (US)

(21) Appl. No.: 14/576,744

(22) Filed: Dec. 19, 2014

(51) Int. Cl. A61F 13/551; B65D 25/14; B65D 43/16; B65D 43/22; B65D 25/04; B65D 25/28

(52) U.S. Cl. CPC A61F 13/5519; B65D 25/04; B65D 25/28; B65D 43/16; B65D 43/22; B65D 25/14; B65D 2525/286

(57) ABSTRACT

A mountable, portable personal hygiene products holder that can be mounted on a surface in a public restroom or other cramped bathroom space, and methods of manufacture and use. The apparatus holds personal feminine hygiene products for discreet transport and also permits easy changes in locations which might otherwise be difficult to maneuver.

### TECHNICAL FIELD OF THE INVENTION

The apparatus of the invention is a portable holder for personal feminine hygiene products and that is usable as a public restroom assistant. The invention can be used to transport feminine personal hygiene products, and also to facilitate use of such products in public restrooms and bathrooms. The invention further comprises methods of manufacture and use.

### BACKGROUND OF THE INVENTION

Menarche, the first menstrual cycle in female humans, begins at approximately age 12-13. This event typically begins a monthly menstrual cycle until the age of menopause, which on the average occurs at about age 62. It is estimated there are about 2 billion women in this age demographic. The need for and the need to use personal feminine hygiene products is consequently a feature of everyday life at least once per month for the major portion of a woman's life. Feminine hygiene products used during menstruation include tampons, sanitary napkins and feminine hygiene cups. Periodically, a change of these hygiene accessories is necessary throughout the day, and the user's location may not be ideal to facilitate such activity.

It is an object of this invention to provide a holder that can conveniently and discreetly store multiple units of feminine hygiene and personal care products such as tampons, undergarment liners, sanitary napkins and pre-wrapped towelettes.

It is a further object of this invention to provide a portable personal restroom assistant that acts as a wall-mountable changing table or shelf for the user while changing feminine hygiene products.

It is another object of the invention to manufacture a portable personal hygiene products holder that has a disposable inner surface that can be changed or cleaned to maintain a hygienic surface for contact with the stored products.

It is a further object of this invention to provide a hands-free means for attending to personal hygiene changes while in a public restroom or small space.

It is yet another object of the invention to provide a personal hygiene products holder for personal use that can hold multiple units of feminine hygiene products of one or more type

### SUMMARY OF THE INVENTION

The portable hygiene products holder of the invention is an accessory that serves as a tampon holder and optionally also a case for other feminine personal hygiene products at the same time. The invention provides both storage and an organizational tool for women, in that it offers women a vehicle for storing multiple types of personal hygiene products such as tampons and sanitary napkins as well as other hygiene aids. It also provides a surface mountable assistant for more easily changing feminine hygiene products such as tampons in a public restroom or other cramped bathroom space.

An advantage of the portable feminine hygiene products holder and restroom assistant of the invention is that it is unobtrusive and compact, provides ample storage, and is also decorative to render it undetectable if displayed publicly. Instead of throwing tampon products randomly, for example, into a purse, women can use the holder of the invention to attractively and privately store and transport those products.

The apparatus comprises a case that includes an upper side and a lower side. Between these two sides is disposed a hinge that enables opening and closure, with a restricted angle of opening to allow the holder to function as a portable changing shelf, for example in a public restroom setting. The upper side includes a recessed receptacle for storage, while the lower side is equipped with a shelf area, loops or tabs for additional storage, and a locking assembly. The invention further includes a retractable handle assembly for hanging the holder. The holder also comprises an integrally attached magnet assembly that allows the holder to be mounted magnetically to a metallic surface, such as a metal door.

### BRIEF DESCRIPTION OF THE DRAWINGS

FIG. 1 is a perspective view of the exterior of a portable personal products holder according to an embodiment of the invention.

FIG. 2 is a perspective view of the interior of a personal products holder according to the invention comprising internal storage receptacles.

FIG. 3 is a side view of an embodiment of the invention showing the relationship of an upper side to the hinge element and to the lower side.

FIG. 4A is a perspective view of a closed holder with the retractable handle extended according to the invention.

FIG. 4B is a planar view of the cover of an embodiment of the holder according to the invention.

FIG. 4C is an exploded view showing parts of the assembly of an embodiment of the invention.

FIG. 4D is a planar front view of an embodiment of the invention.

FIG. 4E is a planar rear view of an embodiment of the invention.

FIG. 4F is a transparent side view of a holder according to the invention showing the movement of the retractable handle in relation to the storage compartment.

FIG. 5A is a perspective view of the underside of the lower side of a holder according to the invention.

FIG. 5B is a planar view of the underside of a holder according to the invention.

FIG. 6 is a perspective view of an embodiment of a closed personal feminine hygiene products holder according to the invention.

FIG. 7 is a side view of an embodiment of a carrying case for the invention which comprises a zipper closure assembly.

FIG. 8 is a perspective view of a carrying case according to an embodiment of the invention that comprises a zipper closure assembly.

FIG. 9 is a perspective view of the rear of a carrying case according to an embodiment of the invention.

### DETAILED DESCRIPTION OF CERTAIN EMBODIMENTS

In preferred embodiments, the apparatus of the invention comprises a feminine hygiene products holder equipped with a hinge having a maximum open angle range of approximately 75° to 100°, preferably about 90°. This angular limitation allows the apparatus to be mounted for use during the process of attending to feminine hygiene needs. In this respect, when in use the holder provides a convenient public restroom accessory that facilitates sanitary hygiene changes. Many public restrooms have metal doors or walls but are not always equipped with shelves where a tampon holder or feminine hygiene products can be laid out for use. The invention eliminates the need for a shelf because the retractable handle enables it to be hung from a hook within the bathroom stall and further stabilized by attachment of the magnetic element to the stall door or wall. The magnetic attachment can also be used without the retractable handle. The holder in this application thus provides a restroom assistant having a suspended, planar changing surface that will allow a woman to open a wrapped feminine hygiene product packet, place the contents on the surface, and organize the contents for ease of use before removal and disposal of the used product. This sequence of events is currently challenging to perform in a public restroom.

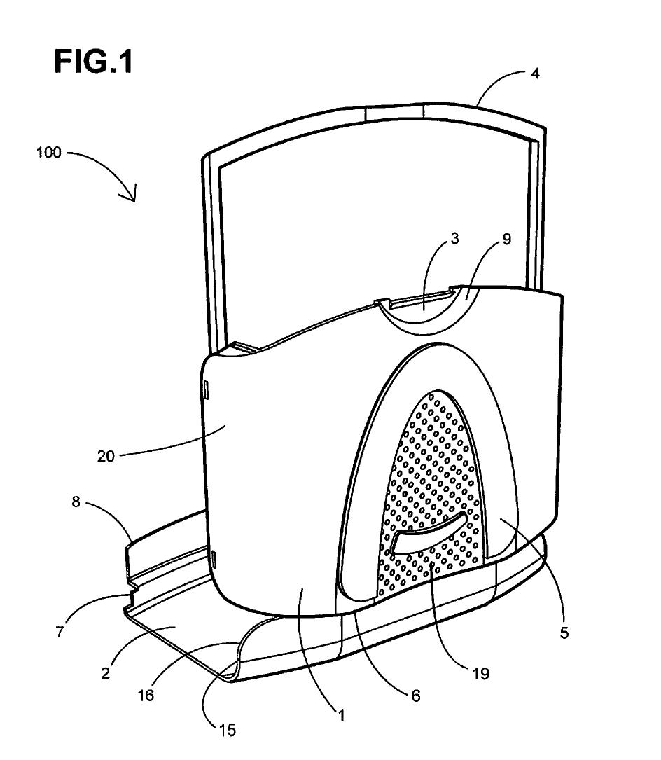

The portable personal hygiene products holder is large enough to hold multiple units of products but small enough to fit into purses of different sizes. According to FIG. 1, a portable personal hygiene products holder according to the invention 100 comprises a cover 1 attached to a base 2 by means of a hinge 6. According to various embodiments of the invention, this holder may be formed of a rigid or semi-rigid material, such as a polymeric substrate material or a metal. In certain other embodiments, a more flexible material such as bendable substrates or woven fabric may be used to construct parts of the apparatus, such as the cover, base or inner compartment walls. As an example, a molded thermoplastic that is infused with an antibacterial or water-repellent material or fiberglass may be used to mold the case and handle parts. This substrate material may be colored or printed with patterns, images, branding or text information. The exterior of the holder may additionally be embossed or superimposed with one or more layers of material, such as fabrics, soft substrates, emblems or plates, thus presenting the appearance of a stylish or personalized accessory case.

The cover 1 further includes an latch closure slot 3 disposed within a detachment recess 9. The purpose of the latch recess 9 allows for easy access to the locking features of the holder while minimizing the risk that the user may break a nail. Further, disposed within the cover 1 is a retractable handle 4 that is housed within the cover 1 when it is not in use.

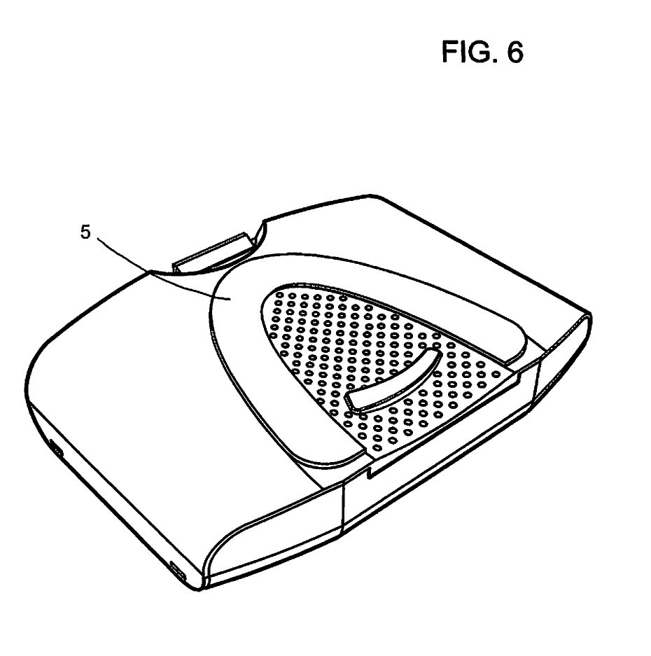

Attached to the outer surface of the cover 1, as shown in FIGS. 1 and 6, is a magnetic element 5. The magnet 5 can be die cut from a flexible magnetic sheet and affixed by conventional means to the back of the cover, or a rigid magnetic element may be used. The flexible magnetic sheeting is typically formed from a magnetized metallic sheet of a thickness of up to 0.1 inch with a tolerance of up to ±0.05 inch. The thickness of the magnetic sheeting typically determines the strength of the magnet. In the invention, the magnetic strength should be sufficient to support the combined weight of the holder and its contents, which may for example range from about 1 oz. to about 8 oz. Solid magnets not made up of layered magnetic and non-magnetic materials may also be used, provided they do not substantially increase the weight of the holder. In the embodiment of FIG. 1, the magnetic element is superimposed over a detachment recess 19 that allows for easy removal of the magnetic from the surface to which it is attached. The recess 19 enables the user to slip a finger(s) beneath the level of the magnet in the recess and lift the magnet away from the surface. One or more decorative elements 23 may also be affixed to the outer surface of the cover 1.

In the exemplary preferred embodiment of FIGS. 1-6, the cover 1 has extended side flanges 20 that form the side walls of the holder 100 to engage with the edges of the base 2 to secure closure of the container.

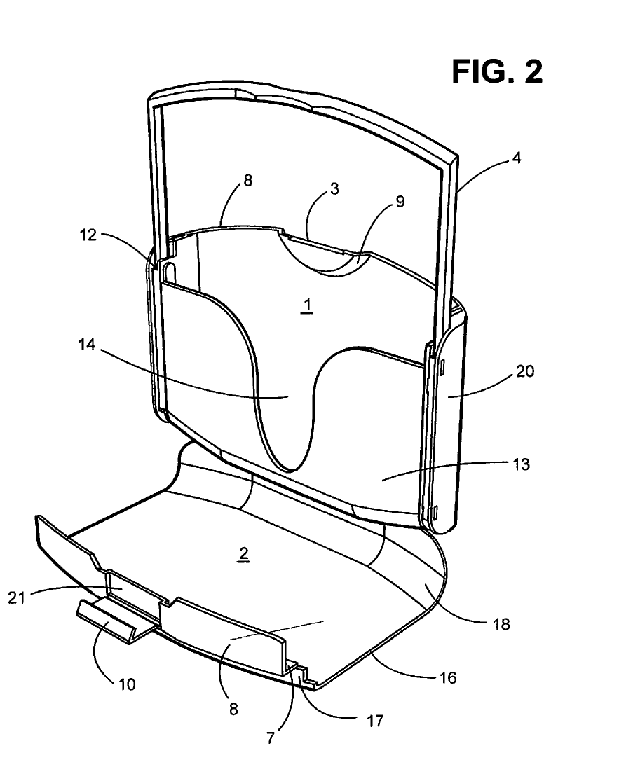

FIG. 2 depicts an internal structure of a portable personal hygiene products holder. Attached to the inside of the cover 1 at the side edges thereof is a receptacle wall 13, which defines a receptacle 14 for storage of the desired personal hygiene products. The receptacle 14 may be of a thickness suitable for holding multiple tampons arranged in planar alignment, for example up to about 1″, preferably up to about ¾″. The edge of the receptacle wall 13 is formed and shaped to allow efficient access to the contents of the receptacle itself, and to allow the viewer to easily view the contents and assess whether the receptacle needs to be restocked. For example, the receptacle wall is curved to allow partial exposure of the tampon contents. One or more additional receptacles (not shown) can be attached to the inner side of the base 2 or be formed by division of the receptacle 14 (also not shown). Tampons and accessories typically used by women for personal hygiene such as wrapped tissues, single units of wrapped, pre-moistened wipes and other sanitary products may also be stored in any of the holder's compartments. The receptacle may be of a size to hold applicator tampons or non-applicator tampons. One or more additional compartments, loops, hooks or adhesive areas can, in other embodiments not shown, be affixed to the internal surface of the base to provide additional storage of varying sizes, shapes and configurations.

A closure lock tab 10 is disposed at the front edge of the cover 1. The holder of the invention is completely closed by engagement of the latch closure slot 3 with the front closure wall 8 and the closure is secure when the contact of these elements moves the closure lock tab 10 outward and away from the holder 100. In the closed position the contact of the side flanges 20 with the base edges 16 form the sides of the closed receptacle. The front of the receptacle is formed by the front closure wall 8 and the front base wall 17. A front receptacle wall 7 extends perpendicularly from the base of the front closure wall 8, thereby forming a stop for the receptacle wall 13 so that it cannot be depressed beneath the level of the closure assembly when the holder is closed. The closure lock tab 10 engages with the recess formed by lower latch element 21 of the front closure wall 8 and fits into a latch closure slot 3 to secure the closure assembly. A slightly acute angle of the latch release 10 in relation to the slot 3 causes the release to be angled slightly downward when it enters the slot, thereby enabling the locking assembly to grip together closely and so resist accidental opening. The closure is opened by finger pressure release activation (e.g. depression and outward pulling) of a latch release 10 that allows the inner edge of the latch release 10 to be withdrawn from the slot 3, thereby allowing separation and opening of the cover and the base.

At the sides of the cover 1, a handle recess 12 is disposed to receive the retractable handle 4. The depth of the recess 12 is compatible with the length of the arms of the handle 4. The shape profile of the handle 4 is formed to align with the profile of the front of the holder 100.

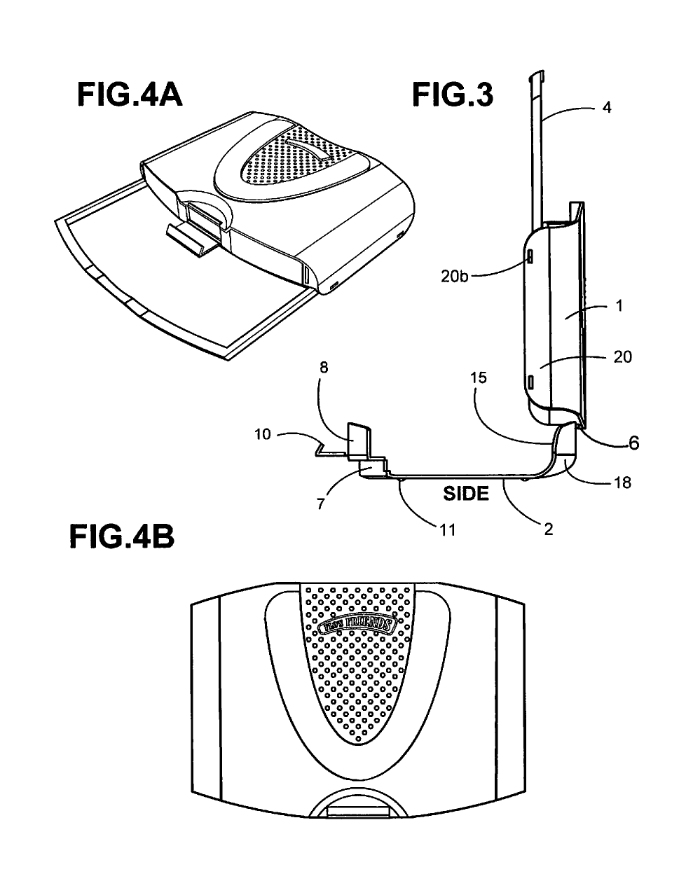

The opening and closure of the holder 100 is accomplished in part by operation of a hinge 6, which is disposed between the cover 1 and the base 2 at the rear receptacle wall 18 of the holder (FIG. 2). As otherwise mentioned herein, the hinge is restricted in its axis of rotation to a maximum range of approximately 90° (meaning that this angle may be up to +20°) so as to allow the base to be perpendicularly disposed when the holder 100 is hung or mounted from a restroom door or wall. As seen in FIG. 3, the hinge is disposed between the lower edge of the cover 1 and the upper edge of a rear edge wall 15. Suitable hinges include case hinges, tray table hinges, card table hinges, barrel hinges or living hinges. A living hinge is an example of a hinge that can be used in certain embodiments. In a thermoformed or molded holder, this type of hinge is typically created of the same material as the cover and base and is formed and adjusted by thinning or cutting to provide the specific appropriate degree of bend to permit opening to the desired angular range.

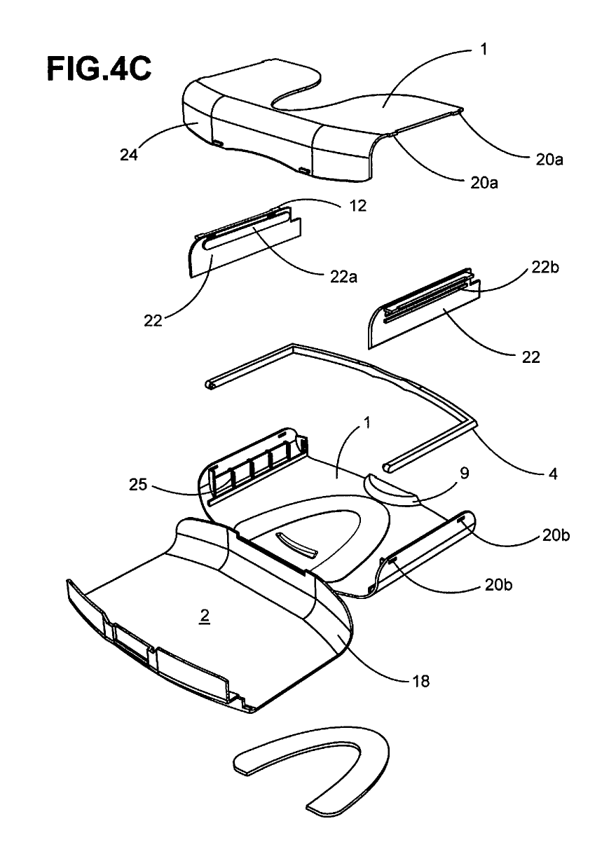

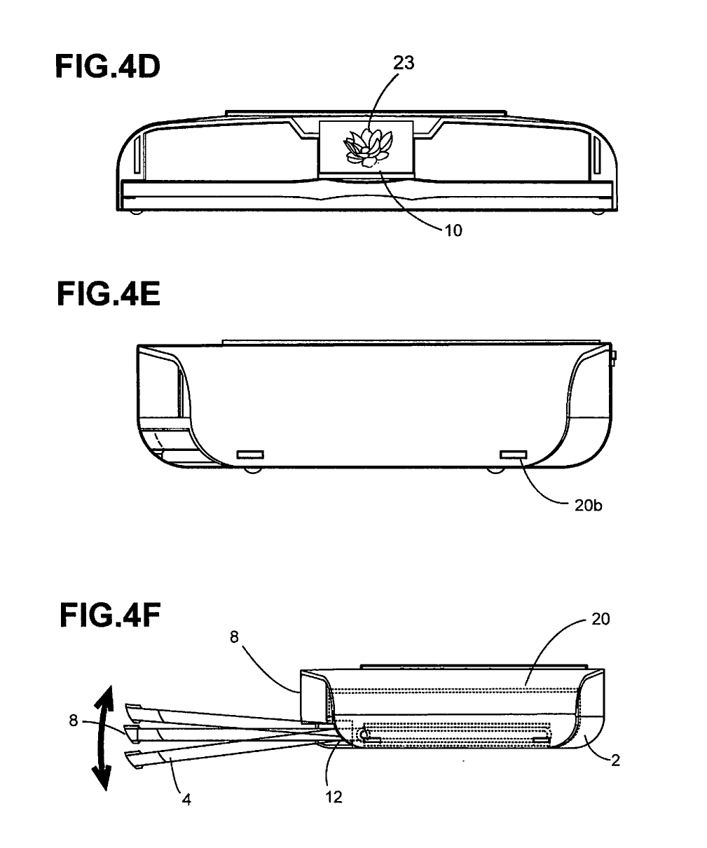

As shown by the embodiment of FIGS. 4A and 4B, the position of the magnetic element 5 circumscribes the detachment recess 19. The recess 19 allows for easier detachment of the holder 100 from the surface to which it is mounted by engagement of the magnet because it prevents a vacuum seal from forming with respect to the entire cover 1.

FIG. 4C provides an exploded view of the assembly of a holder according to one embodiment of the invention. The receptacle wall 13 is formed with a reinforcing receptacle base wall 24 that fits as an inner lining within the rear receptacle wall 18. Tabs 20a engage with slots 20b of the side flanges 20 of the cover 1. Inner receptacle side walls 22 include supports 22b on the external side that contacts the inside of cover side flange 20, and an elongated casement element 22a that with the side wall 22 forms the handle recess 12. Alternatively, the receptacle may be detachable, as described further herein. The receptacle wall 13 engages by friction fit and locking tab and slot closures with the side flanges 20 of the cover to define the internal storage receptacle. The side flange may be further fitted with supports 25 to provide an additional structural stability for the holder 100.

A front, exterior view of an embodiment of the invention is shown by FIG. 4D. Embellishments 23 are variously included, such as embossment affixed to the closure lock tab that can also serve as a frictional contact point to assist with disengaging the closure assembly. FIG. 4E is a side view of the invention. FIG. 4F shows the retractable handle 4 and the movement thereof within the internal handle recess. A limited range of motion is thereby provided that helps with positioning of the holder 100 when it is mounted to a wall surface.

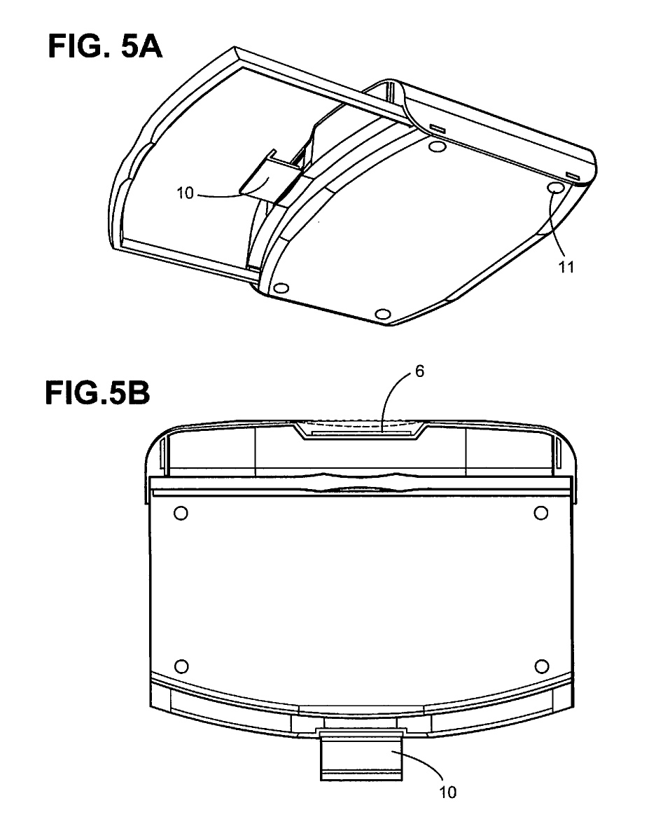

As shown in FIGS. 5A and 5B, the base 2 is affixed with base supports 11 that provide additional support to the holder 100 when it is seated on a horizontal surface. These supports can be constructed, for example, of rubber, plastic or other low skid, nonslip material. This allows the holder to be stably positioned, without slippage on most surfaces when the invention is being used as a restroom assistant. Alternatively, the supports may be integrally molded with the base and serves to provide an elevation of the base from the surface.

The portable personal hygiene products holder of the invention can store multiple units of product, for example up to five tampons. The base 2 may be further compartmentalized into pockets or compartments sized to accommodate wipes, tissues or other sanitary products such as liners or napkins. Preferably, the overall dimensions of the holder are approximately 4″ to about 6″ in length, and approximately 3″ to about 5″ wide, and approximately 0.75″ to about 1.5″ in depth as measured by the depth of the internal cavity when the holder is closed.

In respect to its manufacture, the portable feminine hygiene products holder of the invention may be formed from plastics, polymer composites or metals, all of which provide surfaces that can be easily wiped clean and which are non-absorbent. It may be formed generally in a rectangular or square shape, but it may also for example be thermally molded as a novelty shape. Thermoforming or thermoplastic molding of the components is one cost-effective means of manufacture. In this respect, the parts may be molded separately and assembled, or the manufacturing process may allow for a minimized number of pieces, for example where the cover, side walls, base, inner receptacles and closure assembly are formed as a single piece, with the retractable handle formed as a separate piece.

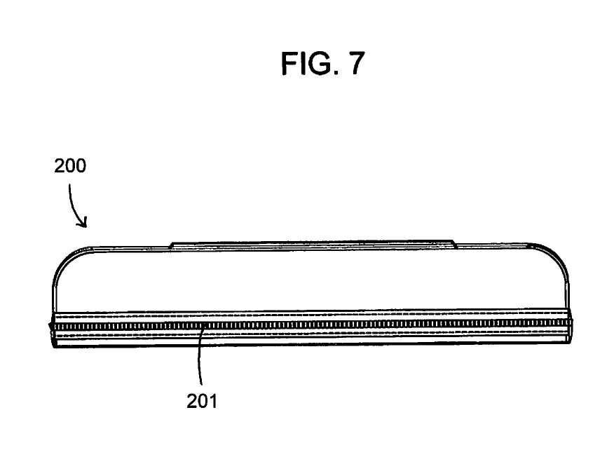

The closure assembly may take alternate forms than are described in the various embodiments presented herein. For example, in one embodiment as shown in FIG. 7, the closure may take the form of a zipper that is attached around the edges of the cover, base and side walls. The zipper may circumscribe three walls and optionally also end segments of the hinge wall.

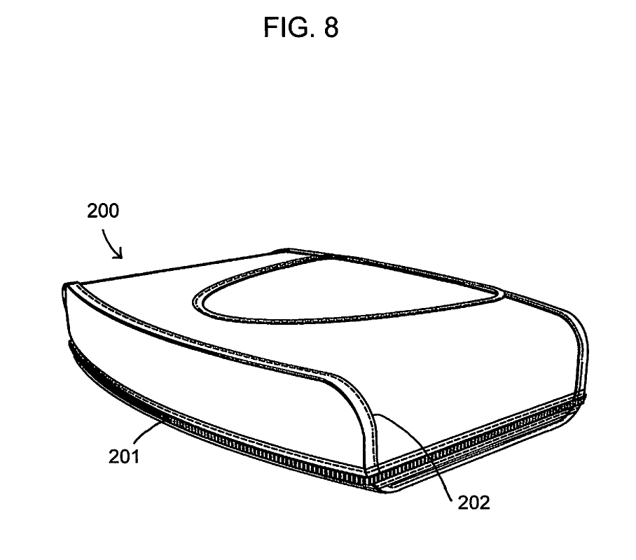

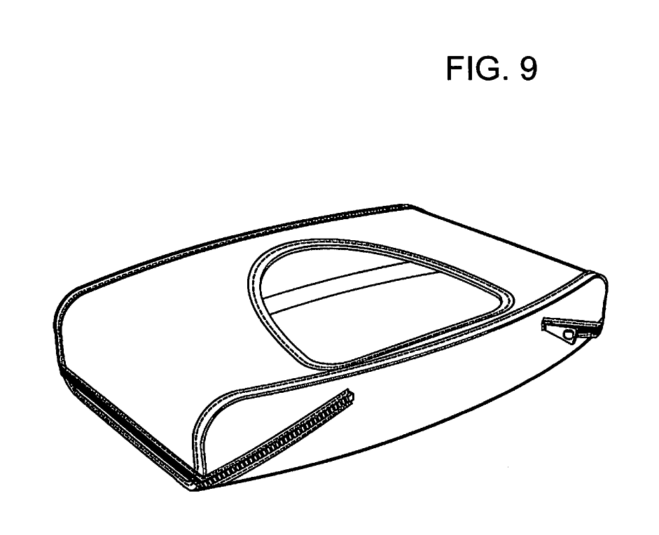

As exemplified by FIGS. 7-9, the portable personal hygiene products holder of the invention may also be carried or stored in a protective case 200, which keeps the holder clean and provides an additional means of discreet storage. This exterior case may be made of any suitable material, including a fabrics or flexible materials, and it may comprise a suitable closure means, such as a zipper. As shown in FIG. 8, a zipper 201 may extend around three sides, or it may as well extend across portions of the fourth side of the perimeter of the exterior case 200, as seen in FIG. 9. In the embodiment shown, the shape of the case is reinforced by stitched seams, however any other method of providing a shape that approximates the shape of the holder 100 stored within the case 200 may be employed.

Also, in other embodiments, as shown in FIG. 8, instead of a retractable handle the outer cover of the portable, feminine personal hygiene products holder of the invention may include a loop for hanging.

In an alternate mode of manufacture, the inner surface of base 2 is lined with a disposable liner material, for example paper or plastic sheeting so that the inner surface can be maintained in a clean, hygienic state over the life of the product. The inner receptacle wall may also be detachable and replaceable, for example with an inner receptacle wall of different size and shape configurations that allow for secure storage of various products of different sizes, such as sanitary napkins and contraceptive devices as well as tampons.

### INDUSTRIAL APPLICABILITY

The invention has applicability in the field of personal feminine hygiene care. The invention is used for discreet storage and transport of hygiene products and also as a device that facilitates hands-free changing of those personal products.

1. A portable feminine personal hygiene products holder comprising a cover that further comprises a latch closure slot and a magnetic element positioned to facilitate attachment of the cover to a vertical metallic surface; a base that is substantially flat and which further comprises a front closure wall, a front receptacle wall, a front base wall, a rear receptacle wall and a latch assembly; an inner receptacle wall attached to the sides of the cover, thereby defining a receptacle inside the cover of a size suitable for storing personal feminine hygiene products; a hinge having a range of motion restricted to approximately 90°; a closure assembly; and a magnet element which has its magnetic surface exposed on the external surface of the cover.

2. The portable feminine personal hygiene products holder of claim 1, wherein the inner receptacle is comprised of compartments of varying sizes.

3. The portable feminine personal hygiene products holder of claim 1 wherein the closure assembly comprises a closure lock tab element that engages operably with a latch closure slot, and which further engages with the front closure wall to form a closure for the holder.

4. The portable feminine personal hygiene products holder of claim 1 which is carried or stored within a protective case.

5. The portable feminine personal hygiene products holder of claim 1 wherein the cover and base are formed from a rigid or semi-rigid material selected from polymeric materials or metals.

6. The portable feminine personal hygiene products holder of claim 1 further comprising one or more compartments, pockets, loops, hooks or adhesive areas attached to the inner surface of the base.

7. The portable feminine personal hygiene products holder of claim 1 wherein the personal hygiene products are selected from the group consisting of tampons, sanitary napkins, wrapped wipes, contraceptive devices and tissues.

8. The portable feminine personal hygiene products holder of claim 1 wherein the base 2 is lined with a removable and disposable liner material.

9. A method of manufacturing a portable feminine personal hygiene products holder comprising forming a cover, a base, a front closure wall, a front receptacle wall, a front base wall, a rear receptacle wall, a closure assembly, and an inner receptacle wall attached to the sides of the cover, thereby defining a receptacle inside the cover of a size suitable for storing personal feminine hygiene products; and further attaching the cover to the base with a hinge having a range of motion restricted to approximately 90°.

10. The method of claim 9 wherein the front closure wall, front receptacle wall, front base wall, rear receptacle wall, closure assembly, and inner receptacle wall by a thermoforming or molding process.

11. The method of claim 9 wherein the front closure wall, front receptacle wall, front base wall, rear receptacle wall and closure assembly are formed as a single piece.

12. The method of claim 9 wherein the inner receptacle wall is formed in multiple, interchangeable configurations to provide storage compartments of varying sizes, shapes and configurations.

13. The method of claim 9 further comprising adding loops, hooks or adhesive areas to provide additional storage.

14. The method of claim 9, further comprising attaching a disposable liner to the inner surface of the base.

15. The method of claim 9 wherein the closure assembly comprises a locking tab and slot assembly or a zipper.

16. A method of storing personal hygiene products for use in a restroom comprising:
a. Providing a holder comprising a cover, a base, a hinge having a maximum range of motion of not more than approximately 90° and an inner receptacle and a closure assembly; wherein the cover further includes a retractable handle, a recessed area for inserting and withdrawing the retractable handle, a latch closure slot and a magnetic element positioned to facilitate attachment of the cover to a vertical metallic surface; and wherein the inner receptacle is formed by an inner receptacle wall attached to the inside of the cover; and further wherein the base includes one or more additional compartments, pockets or loops attached thereto;
b. Storing multiple units of a personal hygiene product in the inner receptacle and the one or more compartments, pockets or loops.

17. The method of claim 16 wherein the personal hygiene products stored in the inner receptacle are selected from the group consisting of tampons, sanitary napkins, wrapped wipes, contraceptive devices and tissues.

18. The method of claim 16 wherein the holder is enclosed within a protective case.
